(19) United States
(12) Patent Application Publication
(10) Pub. No.: US 2022/0117957 A1
(43) Pub. Date: Apr. 21, 2022

(54) TREATMENT METHOD FOR CORONAVIRUS INFECTION

(71) Applicant: Juan Tejada-Duran, South Lawrence, MA (US)

(72) Inventor: Juan Tejada-Duran, South Lawrence, MA (US)

(21) Appl. No.: 17/076,379

(22) Filed: Oct. 21, 2020

(51) Int. Cl. A61K 31/496

(52) U.S. Cl. CPC A61K 31/496

(57) ABSTRACT

A method for treatment of a patient having a coronavirus infection is provided. The method involves treating a patient with a quantify of rifampin. Rifampin has been shown to inhibit the RNA polymerase which coronaviruses rely on for replication/reproduction. Accordingly, treatment with Rifampin has an anti-viral effect, allowing the body's immune system to catch up with and overcome the virus.

### BACKGROUND

### Technical Field

The present disclosure relates generally to antiviral treatment in humans. More particularly the present disclosure relates to a composition which may be used to reduce or inhibit the reproduction of coronaviruses in human tissue.

### Description of Related Art

“Coronaviruses” have a solar corona-like appearance (the surface projections). Coronaviruses are the second most prevalent cause of the common cold (the first are rhinoviruses). An outbreak of severe acute respiratory syndrome (SARS) in 2002 in Guangdong Province, South China spread to Hong Kong, Vietnam, Toronto and then around the world. It was shown that the disease was caused by a coronavirus (SARS-COV). Coronaviruses have also been linked to gastroenteritis in children and adults.

Structure and Replication: Coronaviruses are enveloped virions with the longest positive (+) RNA genome. The glycoproteins on the surface of the envelope appear as club shaped projections that appear as a corona around the virus. The corona formed by the glycoproteins allows the virus to endure the conditions in the gastrointestinal tract and be spread by the fecal-oral route.

The largest positive strand of RNA genome (27,000 to 30,000 cases) associates with N protein to form a helical nucleocapsid. Protein synthesis occurs in two phases similar to that of the Togaviruses. In infection the genome is Translated to produce a polyprotein that is cleared to produce an RNA dependent RNA polymerase (L[225,000DA]). The polymerase generates a negative sense template RNA. The L protein then uses the template RNA to replicate new genomes and produce (5) five to (7) seven individual messengers, ribonucleic acids (m RNA) for the individual viral proteins. Generation of the individual m RNA may also promote recombination events between viral genomes to promote genetic diversity.

Although coronaviruses are most likely transmitted in respiratory droplets, it is also present in sweat, urine, and feces. The novel SARS-CoV-2 virus was shown to be a coronavirus by its electron microscopic morphology and by reverse transcriptase polymerase chain reaction. The novel SARS-CoV-2 virus apparently jumped to man from animals (masked-palm civets, raccoon, dogs and Chinese ferret badgers) raised for food.

Disease Mechanisms of Human Coronavirus:
1) Virus infects epithelial cells of the upper respiratory tract.
2) Virus replicates best at 33° C. to 35° C. Therefore it prefers the upper respiratory tract.
3) Reinfection occurs in the presence of serum antibodies.
4) The glycoprotein corona helps this enveloped virus survive the gastrointestinal tract.
5) Severe acute respiratory syndrome infection is exacerbated by inflammatory responses.

Given the prevalence and infectiousness of coronaviruses, along with the fact that new variants appear to be developing and spreading rapidly, a treatment methodology is desperately needed to bridge the gap until a vaccine may be developed and to treat those who have not been vaccinated for any number of reasons. Using what is known about the mechanics of coronavirus reproduction, it is possible to identify and select potential drugs and treatment regimens to disrupt the virus reproduction in an infected individual. By so disrupting the reproduction, a patient's immune system is given time to catch up to turn the tide and eventually overcome the infection.

### SUMMARY

The subject matter of this application may involve, in some cases, interrelated products, alternative solutions to a particular problem, and/or a plurality of different uses of a single system or article.

In one aspect, a method for treatment of a coronavirus infection is provided. The method involves testing a patient to determine the presence of a coronavirus infection such as a SARS-CoV-2 (COVID-19) infection. If the patient is indeed infected, 300 mg of rifampin is administered to the patient daily for 7-10 days. Following this administering regime, the patient is tested to review a viral load and/or presence of the virus to evaluate the efficacy of treatment and status of the patient. Patient symptoms and severity may also be tracked.

### BRIEF DESCRIPTION OF THE DRAWINGS

FIG. 1 provides a flow chart of one embodiment of the present disclosure.

### DETAILED DESCRIPTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

Generally, the present disclosure concerns a treatment regimen for a patient infected with a coronavirus such as SARS-CoV-2. The regimen includes testing a patient for infection with a coronavirus, and then administering rifampin, monitoring symptoms and then testing again to determine the presence of the infection or not.

As is well known in the art, most viruses encode enzymes essential for viral replication inside a host cell. Viruses use the metabolic machinery of their host cells. Put simply, viruses are obligate intracellular parasites. Viruses can be formed of double or single-stranded DNA or RNA enclosed in a protein coat called a capsid. Some viruses contain a lipid envelop of antigenic glycoproteins.

Usually DNA viruses enter into the host cell nucleus, the viral DNA is transcribed into mRNA by the host cell polymerase. mRNA is translated into virus-specific proteins. An exception of DNA viruses is poxvirus, which has its own RNA polymerase and consequently replicates in the host cell cytoplasm.

In 2002, a severe outbreak of SARS-severe acute respiratory syndrome occurred in Guangdong province, south China. The disease was caused by a coronavirus (SARS-CoV). Coronaviruses measure 80-160 nanometers in diameter. The major human coronavirus proteins are: E2 (peplomeric glycoprotein); E1 (matrix glycoprotein); H1 (hemagglutinin protein); N (nucleoprotein); and L (polymerase).

Coronaviruses have a solar corona-like appearance. They are formed as a single-stranded positive sense RNA genome is enclosed in an envelope containing the E2 viral attachment protein, E1 matrix protein, and N nucleocapsid protein).

Translation of the genome occurs in two phases. The first phase produces an RNA polymerase (L). The second phase, from a negative-sense RNA template, yields structural and nonstructural proteins. The virus then assembles at the rough endoplasmic reticulum of the host cell.

Coronaviruses infect the epithelial cells of the respiratory tract because the optimum temperature for viral growth is 33-35 C. The virus spreads by aerosols and in large droplets. Coronaviruses cause an upper respiratory tract infection similar to the colds caused by rhinoviruses with a longer incubation period (average three days).

In summary, coronaviruses rely on RNA polymerase as a basis for their reproduction. Accordingly, disruption or inhibition of DNA-dependent RNA polymerase will in turn disrupt the reproduction of the coronaviruses of infected cells.

Rifampin (Rifadin, Rimactane) is a semisynthetic derivative of rifampicin B. Rifampin si soluble in organic solvents and in water at acidic pH. Rifampin inhibits DNA-dependent RNA polymerase of micobacteria and other micro-organisms by forming a stable drug enzyme complex leading to suppression of initiation of chain formation (but not chain elongation) in RNA synthesis.

High concentration of rifampin antibiotics can inhibit RNA synthesis in mammalian mitochondria, viral DNA dependent RNA polymerases and reverse transcriptase. Rifampin is bactericidal for both intracellular and extracellular microorganisms.

Therefore, it appears that Rifampin, when administered to a patient infected with a coronavirus, will have anti-viral effects by inhibiting the RNA polymerase on which coronaviruses rely for reproduction. This hypothesis was proven based on testing performed in the Dominican Republic, it has been found that treatment of individuals infected with SARS-CoV-2 with Rifampin has resulted in a much faster recovery times with less severe symptoms.

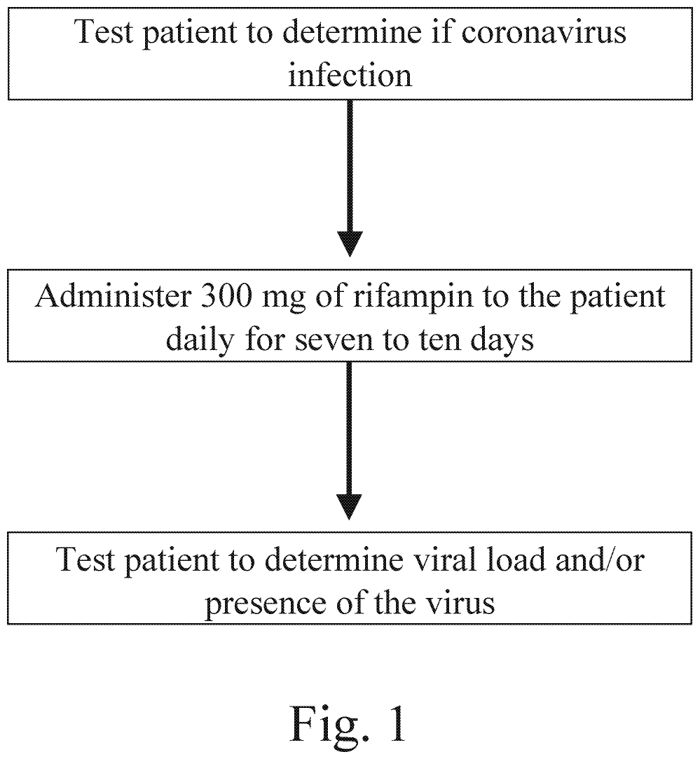

Turning now to FIG. 1, a flow chart of an embodiment of the treatment regime is provided. The first step is testing a patient to determine if there is a coronavirus infection. This testing may be specifically for SARS CoV-2 (COVID-19), or may be for another coronavirus. If an infection is found to be present, the patient is administered Rifampin for 7-10 days. Daily administration quantity may be between 150-400 mg, preferably 300 mg. During the treatment, the patient is monitored closely to track symptoms and treatment progression. In certain embodiments, the patient's viral load may be measured via a tissue or fluid sample. Optimally, this viral load may be seen to decrease over the course of treatment. At the end of the treatment, the patient may be tested again either to measure viral load, or to determine if any infection is present at all.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is:

1. A method for treatment of a coronavirus infection comprising the steps of:
testing a patient to determine the presence of a coronavirus infection;
administering 300 mg of rifampin to the patient daily for seven to ten days; and
after the administering step, testing a tissue or fluid of the patient to determine at least one of the patient's viral load and the presence of the virus.

2. The method of claim 1 wherein the testing step comprises testing to determine the presence of a SARS-CoV-2 infection.
